(19) United States
(12) Patent Application Publication
(10) Pub. No.: US 2019/0000694 A1
(43) Pub. Date: Jan. 3, 2019

(54) Feminine Hygienic Tool Disposal Bag

(71) Applicant: Christy DeGraffenreid, Bakersfield, CA (US)

(72) Inventor: Christy DeGraffenreid, Bakersfield, CA (US)

(21) Appl. No.: 16/024,398

(22) Filed: Jun. 29, 2018

Related U.S. Application Data
(60) Provisional application No. 62/526,800, filed on Jun. 29, 2017.

(51) Int. Cl. A61F 13/551; B65F 1/00

(52) U.S. Cl. CPC A61F 13/5515; A61F 2013/8408; B65F 1/0026

(57) ABSTRACT

A feminine hygienic tool disposal bag is an apparatus that is used to securely contain and deodorize discarded feminine hygiene products. The apparatus includes a container, an elastic rim, and a deodorizing strip. The container is used as a structural unit to mount all of the aforementioned components. The container inverts to ensure a clean exterior for temporary storage of disposed products. The elastic rim widens to enable items to enter the container, to allow the container to invert, and to help secure items in place. The deodorizing strip encircles the container, so that when the user inverts the container, the deodorizing strip is proximal to odors and smells from disposed hygienic articles.

The current application claims a priority to the U.S. Provisional Patent application Ser. No. 62/526,800 filed on Jun. 29, 2017.

### FIELD OF THE INVENTION

The present invention generally relates to a feminine hygienic tool disposal bag that allows users to discreetly hide and dispose of used feminine hygiene products by inverting to prevent the user from having to excessively contact used hygiene items. The present invention further allows users to mask the smell of such items with a deodorizing strip.

### BACKGROUND OF THE INVENTION

For many women, feminine hygiene products are omnipresent and required for regular daily function while ovulation is taking place. Sanitary products may be utilized to contain messes until such a time as the user is able to remove and dispose of them. Presently, the practice of using such items, including tampons, pads, and other extraneous hygienic articles, particularly for discharge or the like, is a discretionary practice, wherein the female user must typically locate herself in a private setting to remove and dispose of the item. This ensures that the items are safely and hygienically contained, and that one person's mess does not become an issue for somebody else.

However, such a system depends on the presence of a means of disposal, which may not be available. The use of conventional containers will not deodorize the hygiene product or even potentially mitigate the leakage of fluid from the extraneous article. Further, many such containers do not invert, meaning that it is easy for a user to accidentally contaminate the outside of the bag. What is needed therefore is a means of containing used feminine hygiene products that serves as a secure and leak-free means of temporary, disposable storage. Further desirable is a device which reduces or eliminates odors associated with such items, while simultaneously replacing undesirable odors with pleasant scents that do not offend.

It is therefore the objective of this invention to provide a bag that meets these requirements. The feminine hygienic tool disposal bag may be flipped inside out in order to keep the user's fingers clean. An elastic rubber closure secures the bag closed at its opening, preventing contents from spilling or exiting the present invention. A rigid base is located at the bottom of the present invention, forming a solid platform against which the user may press to invert the present invention into the closed or full position. A scented band, composed of various scents, rests along the exterior of the bag, so that, upon the bag being flipped inside out, the scented band faces the interior to deodorize and mask the scent of the extraneous article in a compact form that may be flat or rolled up. Thus, the present invention addresses the concerns of a discrete, compact container that may be used to house an extraneous article that emits an odor of varying magnitude.

### BRIEF DESCRIPTION OF THE DRAWINGS

FIG. 1 is a top-right perspective view of the present invention.

FIG. 2 is a top-left perspective view of the present invention.

FIG. 3 is a bottom-right perspective view of the present invention.

FIG. 4 is a top-right perspective detail view of the present invention.

FIG. 5 is a top-left perspective detail view of the present invention.

FIG. 6 is a front view of the present invention with a quantity of liquid fragrance.

FIG. 7 is a front view of the present invention with a fragrance strip.

### DETAILED DESCRIPTION OF THE INVENTION

All illustrations of the drawings are for the purpose of describing selected versions of the present invention and are not intended to limit the scope of the present invention.

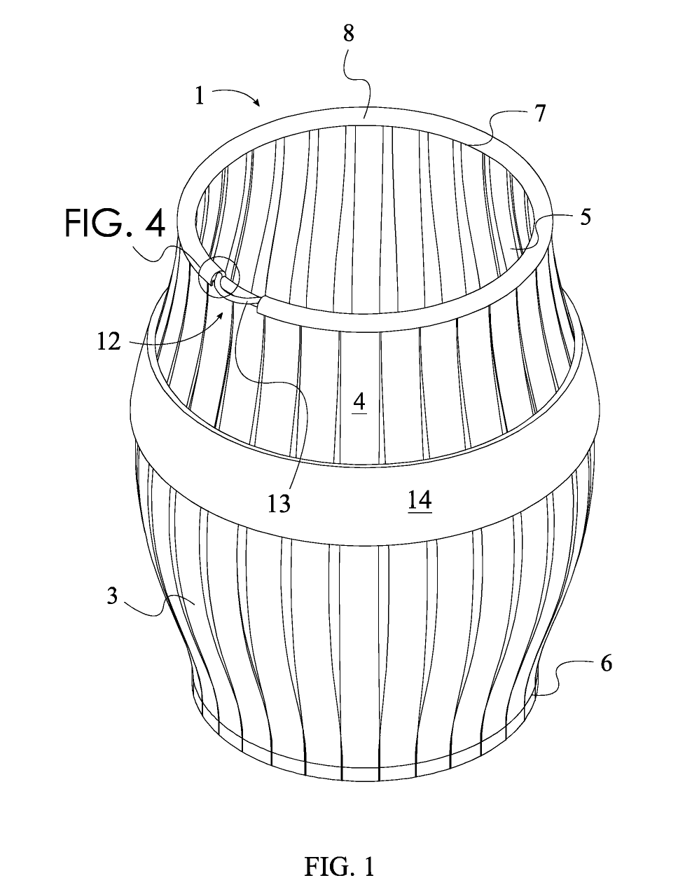

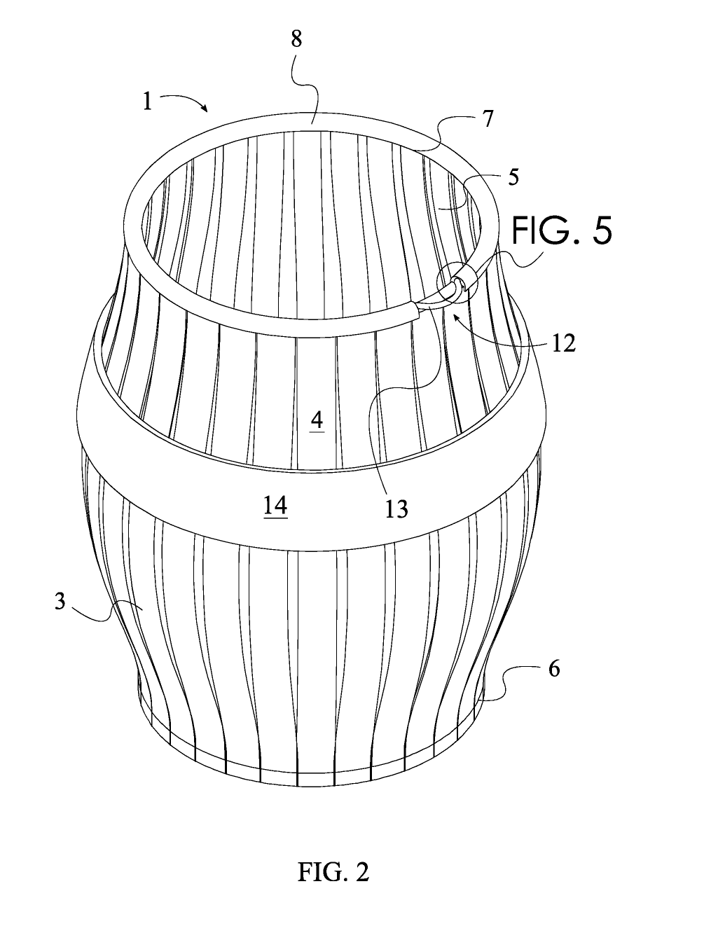

The present invention is a feminine hygienic tool disposal bag that allows a user to contain used hygienic products, particularly monthly feminine care products such as pads and tampons, until a disposal bin becomes available. The present invention utilizes an elastic closure that allows for secure closure, as well as a base 2 that the user can press while grasping the elastic closure in order to invert the bag for sanitary storage in the user's pocket, handbag, or other container. The feminine hygienic tool disposal bag comprises a container 1, an elastic rim 8, and a deodorizing strip 14, as seen in FIGS. 1 and 2. The container 1 is the section of the present invention which, in the preferred usage of the present invention, provides the containment space for used hygienic items. The elastic rim 8 is a closure mechanism through which the container 1 may be pressed in order to invert for item storage. The elastic rim 8 further ensures the secure closure of contained items. The deodorizing strip 14 addresses the common issue of smells or odors which may emanate from used hygiene products by removing those smells from the surrounding air.

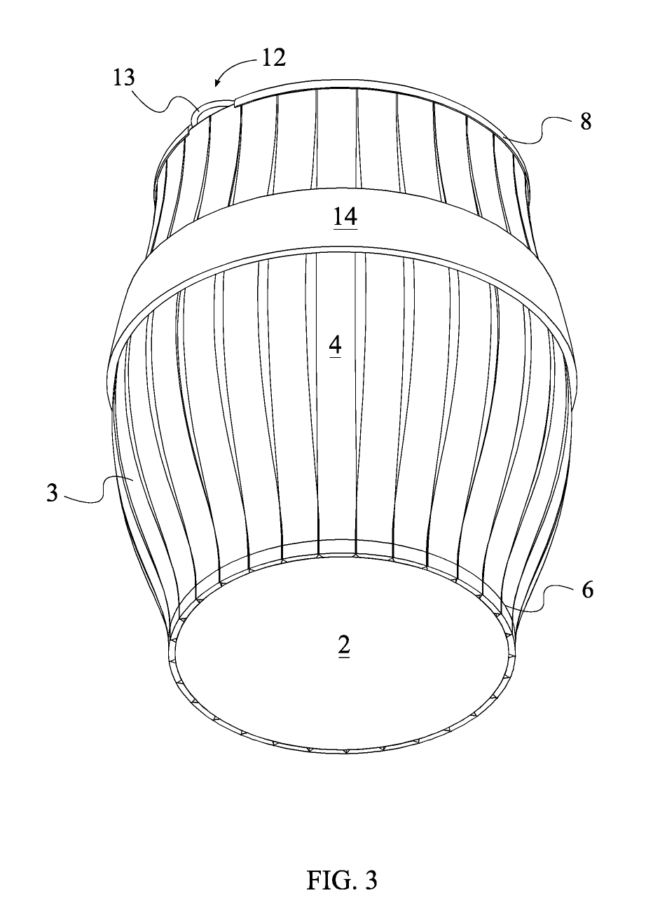

The general configuration of the aforementioned components allows the present invention to efficiently and effectively dispose of used hygienic products when conventional disposal units are not available. The container 1 comprises a base 2 and a tubular wall 3. The base 2 is a generally flexible unit opposite the elastic rim 8 against which a user may press with the user's thumb or thumbs, as seen in FIG. 3. When the user presses the base while pulling the elastic rim 8 with the user's other fingers, the container 1 inverts for item storage. The tubular wall 3 is a hollow cylindrical arrangement of flexible material that connects the base 2 and the elastic rim 8. The tubular wall 3 comprises a first annular surface 4, a second annular surface 5, a first open end 6, and a second open end 7. The first annular surface 4 and the second annular surface 5 are opposing surfaces. The first annular surface 4 is the surface which preferably contacts disposed items. The second annular surface 5 is the surface which inverts to contact the user's pocket, bag or other carrying apparatus. The first open end 6 is the section of the tubular wall 3 opposite the second open end 7 that connects to the base 2. The second open end 7 is the section of the tubular wall 3 opposite the first open end 6 that connects to the elastic rim 8. The first open end 6 is perimetrically connected around the base 2. This arrangement ensures that container 1 contents do not fall out from the container 1 between the first open end 6 and the base 2. The elastic rim 8 is perimetrically connected around the second open end 7. This arrangement constricts the second open end 7 with the elastic rim 8 so that container 1 contents do not fall out from the container 1 between the elastic rim 8 and the second open end 7. The deodorizing strip 14 is connected around the first annular surface 4. In this way, the deodorizing strip 14 is arranged in proximity to the contents of the container 1, particularly when the container 1 is inverted. Furthermore, the deodorizing strip 14 is positioned in between the first open end 6 and the second open end 7. The positioning of the deodorizing strip 14 enables closer proximity of the deodorizing strip 14 to the contents of container 1.

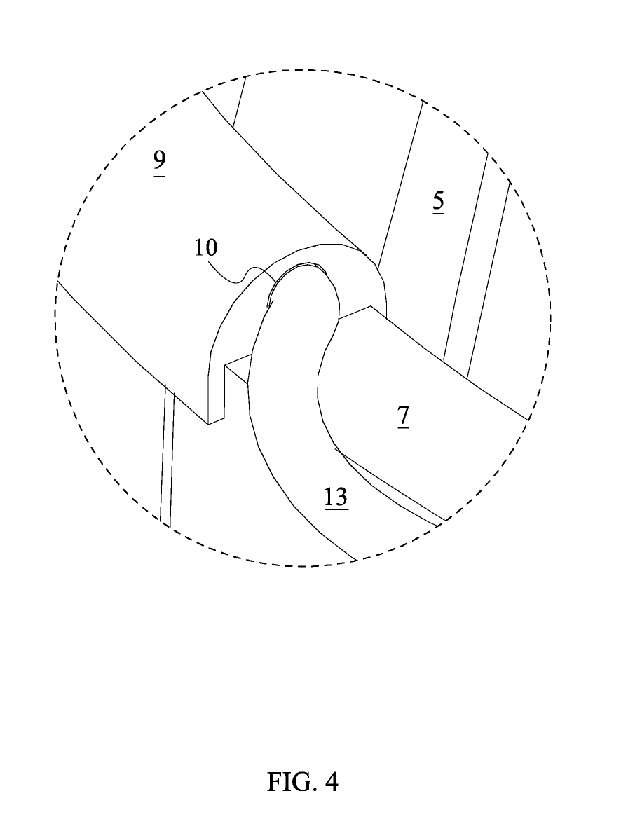

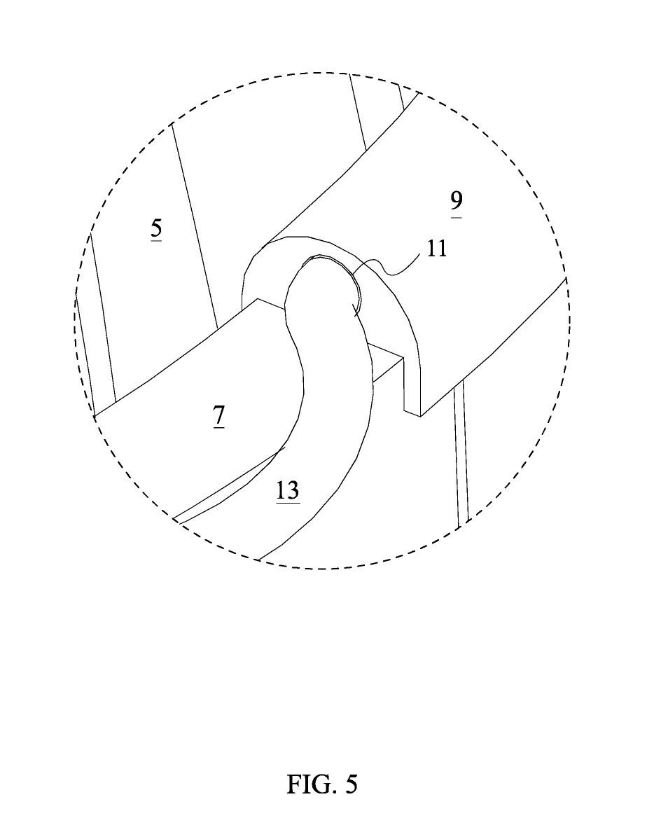

A series of several components within the elastic rim 8 ensures that the user has an acceptable mechanism for closing the container 1 when the container 1 is full. The elastic rim 8 comprises an elastic sleeve 9 and a drawstring 13. The elastic sleeve 9 is used to constrict the second open end 7, while the drawstring 13 is used to cinch the second open end 7. The elastic sleeve 9 is perimetrically connected around the second end. In some embodiments, the elastic sleeve 9 overlaps the first open end 6 and the second open end 7, providing a channel through which the drawstring 13 fits. The drawstring 13 is threaded through the elastic sleeve 9. In an exemplary embodiment, the drawstring 13 is a connected loop of cord, rope, or string. This arrangement of the drawstring 13 relative to the elastic sleeve 9 allows the user to pull the drawstring 13 to tighten the elastic sleeve 9, consequently securing the second open end 7 shut. Furthermore, the elastic sleeve 9 comprises a first sleeve end 10 and a second sleeve end 11, as seen in FIGS. 4 and 5. The first sleeve end 10 and the second sleeve end 11 are positioned offset to each other by an access gap 12. This arrangement allows the user to access the drawstring 13 to tighten the second open end 7. The drawstring 13 traverses the access gap 12, thus providing access for the user to interact with the drawstring 13.

The present invention is arranged to allow for expansion of the container 1 when items are stored within. The first open end 6 is connected perpendicular to the base 2. This arrangement ensures that the tubular wall 3 can expand to adapt to larger items that are placed within the container 1. In addition, the base 2 is a rigid body. This ensures that the user has an intuitive experience when inverting the container 1 for object storage. The tubular wall 3 is arranged in a pleated pattern. The pleated pattern of the tubular wall 3, particularly in conjunction with the perpendicular connection of the first open end 6 to the base 2, allows the tubular wall to expand to accommodate large or multiple items.

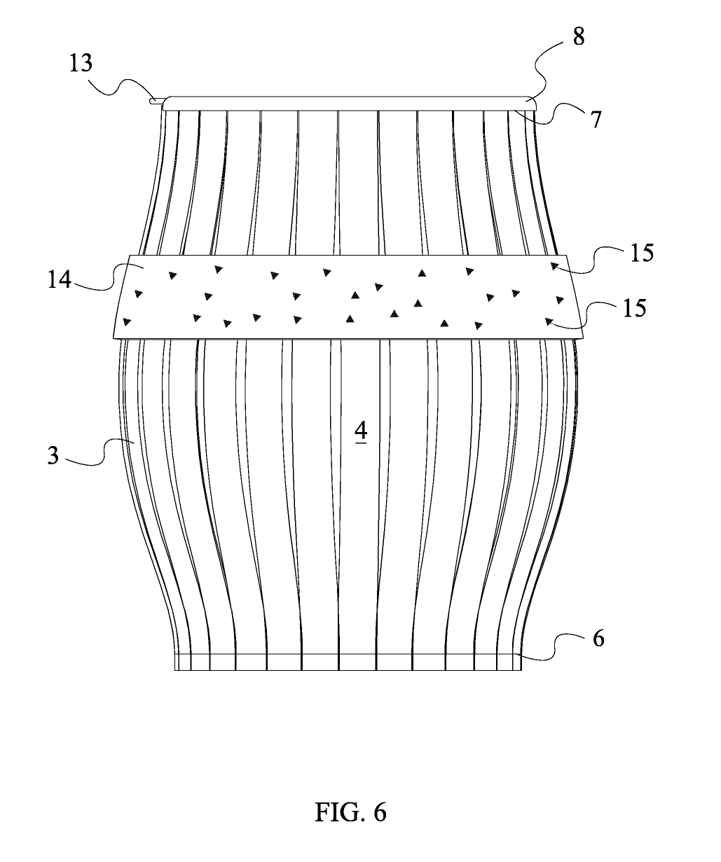

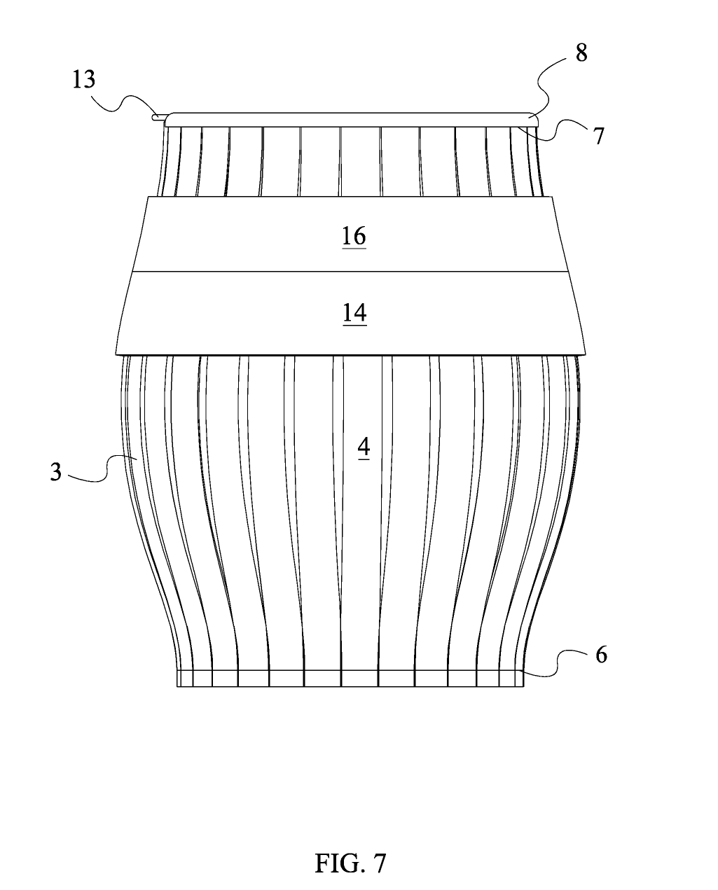

The deodorizing strip 14 is utilized to remove odors from contained products; however, the present invention further generates pleasant scents to mask any excessive odors. In an exemplary embodiment, the present invention further comprises a quantity of liquid fragrance 15, as seen in FIG. 6. The quantity of liquid fragrance 15 is integrated into the deodorizing strip 14, thus enabling the deodorizing strip 14 to simultaneously remove odors and add pleasant scents. Such scents may include, but are not limited to, lavender and cherry scents, as well as other similarly clean and fresh smells. In an exemplary alternative embodiment, the present invention comprises a fragrance strip 16, as seen in FIG. 7. The fragrance strip 16 is connected around the first annular surface 4. This ensures that the fragrance strip 16 will be positioned within acceptable proximity to the contents of the container 1. The deodorizing strip 14 and the fragrance strip 16 are positioned adjacent to each other along the tubular wall 3. In this way, the fragrance strip 16 is optimally positioned to address odors.

In an exemplary embodiment, the container 1 of the present invention is made of an opaque plastic film. This prevents the user from having to view the contents of the container 1, which may be visually unappealing.

Although the invention has been explained in relation to its preferred embodiment, it is to be understood that many other possible modifications and variations can be made without departing from the spirit and scope of the invention as hereinafter claimed.

What is claimed is:

1. A feminine hygienic tool disposal bag comprises:
a container;
an elastic rim;
a deodorizing strip;
the container comprises a base and a tubular wall;
the tubular wall comprises a first annular surface, a second annular surface, a first open end, and a second open end;
the first open end being perimetrically connected around the base;
the elastic rim being perimetrically connected around the second open end;
the deodorizing strip being connected around the first annular surface; and
the deodorizing strip being positioned in between the first open end and the second open end.

2. The feminine hygienic tool disposal bag as claimed in claim 1 comprises:
the elastic rim comprises an elastic sleeve and a drawstring;
the elastic sleeve being perimetrically connected around the second open end; and
the drawstring being threaded through the elastic sleeve.

3. The feminine hygienic tool disposal bag as claimed in claim 2 comprises:
the elastic sleeve comprises a first sleeve end and a second sleeve end;
the first sleeve end and the second sleeve end being positioned offset to each other by an access gap; and
the drawstring traversing across the access gap.

4. The feminine hygienic tool disposal bag as claimed in claim 1 comprises:
the first open end being connected perpendicular to the base.

5. The feminine hygienic tool disposal bag as claimed in claim 1 comprises:
the base being a rigid body.

6. The feminine hygienic tool disposal bag as claimed in claim 1 comprises:
the tubular wall being arranged in a pleated configuration.

7. The feminine hygienic tool disposal bag as claimed in claim 1 comprises:
a quantity of liquid fragrance; and
the quantity of liquid fragrance being integrated into the deodorizing strip.

8. The feminine hygienic tool disposal bag as claimed in claim 1 comprises:
a fragrance strip;
the fragrance strip being connected around the first annular surface; and
the deodorizing strip and the fragrance strip being positioned adjacent to each other along the tubular wall.

9. The feminine hygienic tool disposal bag as claimed in claim 1, wherein the container is made of an opaque, plastic film.

10. A feminine hygienic tool disposal bag comprises:
a container;
an elastic rim;
a deodorizing strip;
a fragrance strip;
the container comprises a base and a tubular wall;
the tubular wall comprises a first annular surface, a second annular surface, a first open end, and a second open end;
the first open end being perimetrically connected around the base;
the elastic rim being perimetrically connected around the second open end;
the deodorizing strip being connected around the first annular surface;
the deodorizing strip being positioned in between the first open end and the second open end;
the fragrance strip being connected around the first annular surface; and
the deodorizing strip and the fragrance strip being positioned adjacent to each other along the tubular wall.

11. The feminine hygienic tool disposal bag as claimed in claim 10 comprises:
the elastic rim comprises an elastic sleeve and a drawstring;
the elastic sleeve being perimetrically connected around the second open end;
the drawstring being threaded through the elastic sleeve;
the elastic sleeve comprises a first sleeve end and a second sleeve end;
the first sleeve end and the second sleeve end being positioned offset to each other by an access gap; and
the drawstring traversing across the access gap.

12. The feminine hygienic tool disposal bag as claimed in claim 10 comprises:
the first open end being connected perpendicular to the base.

13. The feminine hygienic tool disposal bag as claimed in claim 10 comprises:
the base being a rigid body.

14. The feminine hygienic tool disposal bag as claimed in claim 10 comprises:
the tubular wall being arranged in a pleated configuration.

15. The feminine hygienic tool disposal bag as claimed in claim 10, wherein the container is made of an opaque, plastic film.

16. A feminine hygienic tool disposal bag comprises:
a container;
an elastic rim;
a deodorizing strip;
the container comprises a base and a tubular wall;
the tubular wall comprises a first annular surface, a second annular surface, a first open end, and a second open end;
the first open end being perimetrically connected around the base;
the elastic rim being perimetrically connected around the second open end;
the deodorizing strip being connected around the first annular surface;
the deodorizing strip being positioned in between the first open end and the second open end;
the elastic rim comprises an elastic sleeve and a drawstring;
the elastic sleeve being perimetrically connected around the second open end;
the drawstring being threaded through the elastic sleeve;
the elastic sleeve comprises a first sleeve end and a second sleeve end;
the first sleeve end and the second sleeve end being positioned offset to each other by an access gap; and
the drawstring traversing across the access gap.

17. The feminine hygienic tool disposal bag as claimed in claim 16 comprises:
the first open end being connected perpendicular to the base; and
the base being a rigid body.

18. The feminine hygienic tool disposal bag as claimed in claim 16 comprises:
the tubular wall being arranged in a pleated configuration.

19. The feminine hygienic tool disposal bag as claimed in claim 16 comprises:
a quantity of liquid fragrance; and
the quantity of liquid fragrance being integrated into the deodorizing strip.

20. The feminine hygienic tool disposal bag as claimed in claim 16 comprises:
a fragrance strip;
the fragrance strip being connected around the first annular surface; and
the deodorizing strip and the fragrance strip being positioned adjacent to each other along the tubular wall.
